(19) United States
(12) Patent Application Publication
(10) Pub. No.: US 2026/0137331 A1
(43) Pub. Date: May 21, 2026

(54) METHOD FOR CLASSIFYING ELECTROENCEPHALOGRAPHY DATA WITH THE USE OF ARTIFICIAL NEURAL NETWORKS

(71) Applicant: UNIVERSITÄT BERN, Bern (CH)

(72) Inventors: Florence Marcelle AELLEN, Wabern (CH); Athina TZOVARA, köniz (CH)

(21) Appl. No.: 19/119,078

(22) PCT Filed: Oct. 17, 2023
(86) PCT No.: PCT/IB2023/060473

§ 371 Date: Apr. 7, 2025

(30) Foreign Application Priority Data
Oct. 20, 2022 (EP) 22386068.5

(51) Int. Cl. A61B 5/38; A61B 5/00

(52) U.S. Cl. CPC A61B 5/38; A61B 5/7267

(57) ABSTRACT

A sensor system is proposed in one example for analysing electroencephalography sensor signals obtained by a plurality of sensors connected to an object to be stimulated with a sequence of stimulation signals. The sensor system comprises means for: acquiring a set of sensor signals in response to stimulating a plurality of objects with a plurality of sequences of stimulation signals comprising a set of standard stimulation signals and a set of deviant stimulation signals; pre-processing the acquired set of sensor signals to obtain a set of pre-processed sensor signals; preparing an artificial neural network for data analysis by using a first set of the pre-processed sensor signals, the preparation comprising the steps of training and validating the network by using the first set of the pre-processed sensor signals; feeding a second set of the pre-processed sensor signals to the network as trained and validated; and combining output signals of the network as trained and validated in response to feeding the second set of the pre-processed sensor signals to the network to obtain a single classification result for a respective object and for a respective sequence of stimulation signals.

### FIELD OF THE INVENTION

The present invention relates to a data classification method, which according to one example may be used for predicting awakening of a comatose patient. The method involves using an artificial neural network (ANN) to generate a classification result. The present invention also relates to a sensor system and an artificial neural network for implementing the steps of the method.

### BACKGROUND OF THE INVENTION

Electroencephalography (EEG) is a technique that measures activity of the human brain via electrodes positioned on the scalp. EEG is commonly used for research and clinical purposes, for example to study neural responses to stimuli of the environment, such as sounds.

Comatose patients, e.g., after a cardiac arrest, admitted to intensive care units of hospitals undergo a multitude of assessments of clinical variables and physiological signals. EEG is routinely collected at patients' bedside and can provide information about the integrity of neural functions, which may be used to anticipate the patients' outcome from coma. The EEG measurement in the clinical routine is offline evaluated by clinicians, which is time consuming and prone to subjectivity. Moreover, existing clinical markers for prognosticating outcome from coma may leave up to one third of patients with an indeterminate prognosis.

One marker for estimating the integrity of neural functions in coma is related to auditory processing. Patients with a more intact auditory processing have been shown to be more likely to survive. The auditory process is evaluated most commonly with at least two different types of sounds, one repeated (standard) and one scarce (deviant) sound. The typical way to analyse EEG signals received from electrodes on the scalp is by averaging hundreds of EEG responses to the same sound together and investigating the amplitude or latency of the auditory responses, or difference between responses to standard and deviant sounds. The features used for prediction of coma outcome are therefore selected a priori and might neglect important characteristics of the auditory response. Overall standardised auditory stimulations are not currently routinely used in the clinics.

Other approaches to differentiate neural processing of auditory stimulations with regards to standard and deviant sounds extract EEG patterns, which are specific for a patient and are modelling single-trial EEG responses (e.g., Tzovara A, Rossetti A O, Spierer L, et al., “Progression of auditory discrimination based on neural decoding predicts awakening from coma”, Brain 2013; 136 (1):81-89). These studies showed that a difference in auditory discrimination between EEG data recorded from the first to the second day can help to anticipate patients' outcome, but require two different EEG recordings, on two consecutive days. Later studies on a similar cohort showed that neural synchrony across the scalp with respect to standardised auditory stimulations is informative of the chances of awakening for these patients. However, to date, there is not an automated machine learning-based technique that can analyse responses to sounds in coma, based on one single EEG recording, and anticipate patients' chances of awakening.

As machine and deep learning tools have become more powerful in recent years, they have also been used for analysing brain signals. Artificial neural networks (ANN) have been used for a multitude of tasks, as they allow for a feature extraction in a data-driven way and may outperform traditional techniques. However, it remains unknown how to apply these networks on data recorded in coma patients, in response to sounds, with the application of anticipating patients' outcome.

Other studies that have used ANNs to analyse EEG data and anticipate patients' outcome have used continuous recordings of EEG (e.g., Tjepkema-Cloostermans M C, da Silva Lourenço C, Ruijter B J, et al. “Outcome Prediction in Postanoxic Coma With Deep Learning”, Crit. Care Med. 2019; 47(10):1424-1432). These studies show high performance in anticipating patients' outcome, but they do not provide additional information with respect to the current clinical tests, because they rely on the same signals that are currently used in the clinics. The present invention uses ANNs on recordings of EEG signals in response to auditory stimulation. This can provide complementary information about anticipating the patients' outcome, because to date, standardised auditory stimulation is not used in the clinical routine.

US2021022638A1 discloses a method for generating an indicator of the state of a patient in coma including: generating at least one auditory stimulation by generating a sequence of auditory stimuli, the sequence producing evoked potentials in the patient; acquiring a first electroencephalographic signal produced by patient from at least one electrode; estimating at least one pair of values corresponding to a first parameter and a second parameter extracted from the first acquired signal, including estimating a first pair of values such that calculating the first parameter includes an estimation of the amplitude variance of the first signal within a predefined time window and the calculation of the second parameter includes an estimation of the correlation of two segments of the first signal; generating a state indicator for the or each pair of values of the first and second parameters, the values defining coordinates of a point in a reference base.

Publication “Predictive analysis of patient recovery from cardiac-respiratory arrest” by Floyrac A. et al., bioRxiv, 27 May 2019 (2019-05-27), XP93031191 presents a method to predict the return to consciousness from post-anoxic coma of hospitalised patients based on the analysis of periodic responses to auditory stimulations, recorded from surface cranial electrodes.

Publication “EEGNet: A Compact Convolutional Neural Network for EEG-based Brain-Computer Interfaces” by Vernon J. Lawhern et al., arxiv.org, Cornell university library, 201 Olin Library Cornell University ITHACA, NY 14853, 23 Nov. 2016 (2016-11-23), XP081354609 introduces a compact convolutional neural network for EEG-based brain computer interfaces. The publication introduces the use of depthwise and separable convolutions to construct an EEG-specific model which encapsulates well known EEG feature extraction concepts for brain computer interfaces.

### BRIEF DESCRIPTION OF THE INVENTION

The present invention aims to overcome at least some of the above-identified problems. More specifically, present invention proposes a novel solution for analysing sensor signals by using an ANN. The proposed solution may for instance be used for predicting awakening of a comatose patient. However other applications are also possible.

According to a first aspect of the invention, a sensor system for analysing sensor signals is provided as recited in claim 1.

When applied to predicting awakening of comatose patients, the proposed solution has the novelty that it uses standardised auditory stimulations together with an ANN to anticipate the outcome of comatose patients. The proposed solution has the following advantages with respect to existing techniques: (a) the anticipation of coma outcome is based on one single EEG recording (as opposed to two or more recordings, performed in two consecutive days), which makes the method easier to implement on prospective patients; (b) the process can be fully automatic, and provides quantitative and objective output (as opposed to current markers of coma outcome in the clinics which rely on visual expert scorings and can be prone to subjectivity); (c) it is based on an ANN which is a powerful yet not widely used technique for analysing EEG responses to sounds, and which requires minimal assumptions and preparation of the data (as opposed to solutions requiring a selection of features based on a priori knowledge); (d) it is based on one ANN that can be pre-trained with EEG data of retrospective coma patients and then applied on data of prospective patients, with minimal processing time per prospective patient (as opposed to some solutions requiring the calculation of one computational model per prospective patient and EEG recording, which is time consuming); (e) it is based on standardised auditory stimulation, which is currently not part of the clinical evaluations of these patients, and thus provides additional information on patients' outcome, including information for patients that would have indeterminate outcome prognosis based on existing clinical criteria (as opposed to for example the above-mentioned publication “Outcome Prediction in Postanoxic Coma With Deep Learning”, or similar ones, which also anticipate outcome with the use of ANNs, but based on EEG recordings without auditory stimulation, which are currently used in the clinics and thus give comparable outcome predictions with trained experts).

According to a second aspect of the invention, an artificial neural network for analysing sensor signals obtained by a plurality of sensors connected to an object to be stimulated with a sequence of stimulation signals is provided as recited in claim 11.

According to a third aspect of the invention, a method for analysing sensor signals obtained by a plurality of sensors connected to an object to be stimulated with a sequence of stimulation signals is provided as recited in claim 14.

According to a fourth aspect of the invention, there is provided a computer program product comprising instructions for implementing the steps of the method when loaded and run on a computing apparatus or an electronic device.

Other aspects of the invention are recited in the dependent claims attached hereto.

### BRIEF DESCRIPTION OF THE DRAWINGS

The invention will now be described in more detail with reference to the attached drawings, in which:

FIG. 1 schematically illustrates a network architecture showing an artificial neural network according to an example of the present invention;

FIG. 2 schematically illustrates the process of predicting awakening of a comatose patient with the use of the artificial neural network of FIG. 1;

FIG. 3 shows some example results when implementing the process by using the artificial neural network of FIG. 1, obtained from a group of 134 coma patients, showing that the process by using the artificial neural network is sensitive in predicting patients' outcome, and in particular their chances of awakening from the coma; and

FIGS. 4a to 4c show a flowchart illustrating the data classification method according to an example of the present invention.

### DETAILED DESCRIPTION OF THE INVENTION

It should be noted that the figures are provided merely as an aid to understanding the principles underlying the invention, and should not be taken as limiting the scope of protection sought. As used herein, unless otherwise specified the use of the ordinal adjectives “first”, “second”, “third”, etc. to describe a common object, merely indicate that different instances of like or different objects are being referred to, and are not intended to imply that the objects so described must be in a given sequence, either temporally, spatially, in ranking, or in any other manner. As utilised herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.” Furthermore, the term “comprise” is used herein as an open-ended term. This means that the object encompasses all the elements listed, but may also include additional, unnamed elements. Thus, the word “comprise” is interpreted by the broader meaning “include”, “contain” or “comprehend”.

The present invention is next described in more detail in the context of predicting awakening of a comatose patient, but the invention is not limited to this application. More broadly, the present invention proposes a data classification system configured to classify sensor signals into different target groups in response to stimulating an object with a set of stimulation signals, where the stimulation signals are audio signals in the embodiment explained below in more detail. The sensor signals are generated by a set of sensors connected to the object to be stimulated. In the embodiment explained below, the sensors are electrodes of an electroencephalography (EEG) system. The method as explained below involves exposing a comatose patient after cardiac arrest, to auditory stimuli within the first 24 hours from the onset of coma. The auditory stimuli comprise repeated standard and deviant sounds as explained later in more detail. The method then involves recording the patient's electrical activity in the form of EEG data to measure the auditory evoked potential (AEP) for standard sounds and for deviant sounds of the auditory stimuli. The electrical activity signals are pre-processed, and subsequently a trained ANN is used to analyse the pre-processed electrical activity signals.

Comatose patients, after a cardiac arrest and in the intensive care unit of hospitals are presented with a sequence of audio signals, referred to as sounds, at their bedside, during which their brain activity is measured with a clinical EEG machine. In this example, the sound sequences consist of pure tones, such as 16-bit stereo sounds, sampled at a given sampling frequency, for example at 44.1 KHz, although other sounds are also possible. Linear amplitude envelope of for example 10 ms is applied to the sounds at the sounds' onset and offset. Between sounds an interstimulus interval, which in this example is fixed, is used. The stimulus interval is a silent interval. In this specific example the length of the stimulus interval of 700 ms is used.

In the present example, four different sounds are included in the protocol, namely standard sounds, which are commonly repeated, such as in 70% of the total presentations, and deviant sounds, such as in 30% of the total presentations. The deviant sounds differ from the standard sounds in terms of their duration, pitch, and/or location. The standard sounds have a pitch of, for example 1000 Hz and a duration of 100 ms. Duration deviant sounds differ in terms of duration of the sound presentation, which may be for example 150 ms. Location deviant sounds have an interaural time difference. In other words, one of the ears leads with a given time duration, such as 700 μs. Pitch deviant sounds have a pitch of 1200 Hz, for example. The sound sequence in this example includes a pseudo-randomised ordering of these sounds, consisting of at least 50 sounds, or in particular of at least 100, or 1500 sounds. In this specific example, the sound sequence consists of 1500 sounds. Data can be recorded with different electrode setups consisting of 5 to 63 electrodes depending on the implementation, and with a suitable sampling frequency of at least 80 Hz or more specifically of at least 500 Hz for analysing EEG signals outputted by the electrodes. In this specific example, the sampling frequency of 1000 or 1200 Hz is used.

The pre-processing steps of the EEG signals are next explained in more detail. The pre-processing steps are performed prior to feeding the EEG signals as processed to the artificial neural network (ANN). The processing pipeline comprises the following steps, not necessarily strictly in that order. The EEG signals are referenced to a reference signal by subtracting for every time point, (i.e., sampling instant) the mean voltage over all electrodes from the respective EEG signal. The EEG signals are also filtered between for example 0.1 and 40 Hz. Other reference and/or filter settings are also or instead possible. Around every auditory stimulation an interval of EEG activity, for example −50 ms to 500 ms is extracted, where the value of −50 ms means that the extracted time period begins 50 ms before the respective sound begins. These in time domain cropped EEG signals from all EEG electrodes are in the following called “single-trials”. In other words, the cropped EEG signals collectively from all electrodes connected to a given patient per sound are referred to as single-trials, which thus form a combined EEG signal. Artefacts of, for example ±100 uV, are rejected across all or any of the electrodes, although other artefact rejection approaches can also or instead be used. Artefacted (e.g., noisy) or missing signals (electrodes) may be interpolated using standard techniques, such as three-dimensional (3D) splines. In other words, the interpolations may be performed according to the activity of the neighbour electrodes. A number of electrodes, at least 5 is selected for the analysis and the signals are downsampled to a sampling frequency of 1000 Hz, for instance, although other sampling frequencies are also possible. The single-trials may further be baseline-corrected, and the EEG signals obtained in response to stimulating the patient with a number of sounds (standard and deviant) are used for further analysis. The last step of the pre-processing is in this case the normalisation of the EEG signals for each single-trial to a mean of zero and standard deviation of one.

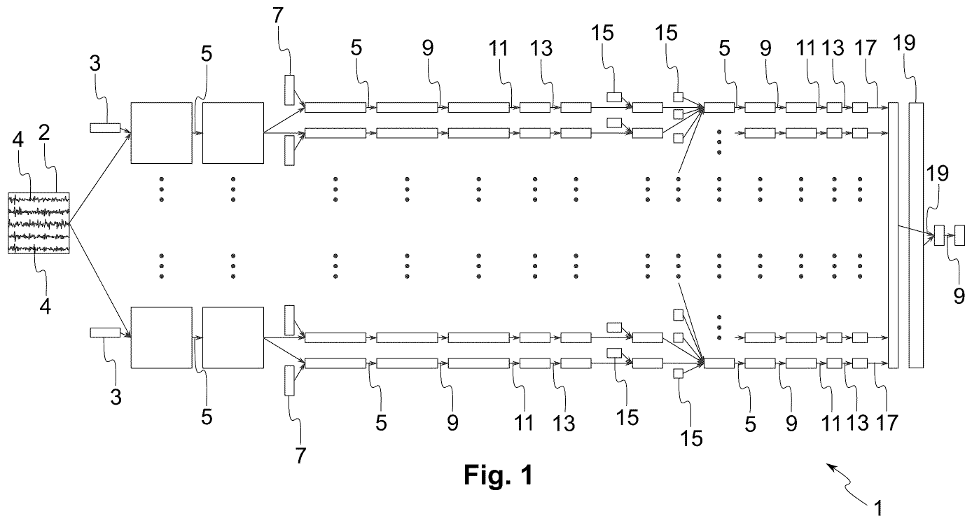

FIG. 1 schematically illustrates the ANN 1, which is a convolutional neural network, according to an example of the present invention. The architecture shown in FIG. 1 merely illustrates one example network architecture, but the network may be varied in many ways as becomes clear by reading the following description. The architecture as shown in FIG. 1 consists of 15 functional or network layers, where the first one is a convolutional layer 3. We input a set of signals 2 (which are composed of a set of pre-processed EEG signals) with the shape (b, c, tp, 1) into the ANN 1, where b denotes the batch size, i.e., the number of single-trials used to train the network simultaneously, c is the number of EEG electrodes or signals, and tp the number of time points, i.e., the length of the signal in seconds times the sampling frequency (see above). In the present description, the dimension of the signal refers to a 2D signal (x, y), 3D signal (x, y, z) or 4D signal (v, x, y, z), etc. With the size or shape of the signal it is referred to the number of datapoints per dimension, e.g., a signal of size (1, 137, 64) has in the first dimension a size of 1, in the second dimension a size of 137 and in the third dimension a size of 64. The convolutional layer has a kernel of size (1, k), meaning that only neighbouring timepoints with a maximal distance of k are able to interact with each other. The different EEG signals 4 are not able to exchange any information. The output of the first layer is of shape (b, c, tp, f1), where f1 denotes the number of filters in the convolutional layer, which is equivalent to the number of kernels used. The convolutional layer is thus configured to convolve a respective EEG signal 4 only along the temporal dimension, but not along the spatial dimension. Furthermore, in this example, the number of filters in the convolutional layer is greater than 16. It is to be noted that respective output/input signals are schematically illustrated with rectangles between two consecutive layers in FIG. 1.

The second layer is a batch normalisation layer 5, which is used to standardise the signals at this stage to a mean of zero and standard deviation of one, over the whole batch. The third layer is a two-dimensional (2D) depthwise convolutional layer 7, with a kernel size of (c, 1). This layer convolves the input with a kernel per filter separately and since the second argument of the kernel size was set to 1 the multiplication of this layer only considers information for a single time point simultaneously but allows information of different EEG electrodes to influence one another. In this example, the kernel of this layer has a spatial dimension that is the same as the number of electrodes connected to the object. An additional parameter for this layer is the depth multiplier d, deciding how many different new filters are built per filter from the input of this layer. The output signal of this layer has therefore the shape (b, 1, tp, f1*d). The 2D depthwise convolutional layer 7 is thus configured to reduce the size of the input signals of this layer (and in particular the size of the second dimension of the input signal), and the depth multiplier has a value of at least 4 in this example.

The 2D depthwise convolutional layer 7 is followed by a fourth layer, namely another batch normalisation layer 5, and a fifth layer, which is an activation layer 9, where an activation function is applied to the signal. In this example, the activation function is func. Both of these layers do not have an influence on the size of the signal.

The sixth layer is a pooling layer 11, which is used to decrease the size of the signal, here by averaging a patch of the signal (here of size (1, 4)) together. In other words, this layer calculates the mean in an area of the “pool size”, and reduces the size of the signal. The signal is thus in this example decreased to a size of (b, 1, tp/4, f1*d) once it passes through this layer.

The seventh layer is a dropout layer 13, where a percentage dr of the signal is randomly dropped. All the dropped values are set to zero and they do not further contribute to the final prediction.

The following eighth layer is the final convolutional layer, which is a 2D separable convolutional layer 15, consisting first of a depthwise convolution and then a pointwise convolution. The depth convolution with a given kernel size, in this example of size (1, 16), convolves only along the time dimension with the depth multiplier set to 1, giving an output of (b, 1, tp/4, f1*d). The pointwise convolution allows for interaction of the signal across different filters. The number of output filters is set to f2 such that the output signal from this layer will have a shape of (b, 1, tp/4, f2). This layer is again followed by a second batch normalisation layer 5 (ninth layer), a second activation layer 9 (tenth layer), a second pooling layer 11 (eleventh layer with a pool size=(1, 8)) and a second dropout layer 13 (twelfth layer with a dropout rate=dr). The size of the signal is in this example reduced to (b, 1, tp/32, f2) by the pooling layer.

The thirteenth layer is a flattening layer 17, where the input signal of this layer is flattened, and its dimension is reduced to one per single-trial, leaving the signal to have a size of (b, tp/32*f2). The fourteenth layer is a densely-connected layer 19, connecting all input nodes of the signal together, and allowing interactions between them. The number of output 5 nodes for this layer is chosen to be the same as the number of output classes for the full pipeline (dout) and the signal is therefore reduced to a size of (b, dout). In other words, the dimension of the signal was reduced to one in the flattening layer and has then per batch for example a size of 1088 datapoints. The densely-connected layer (or the last one of these layers if there are more than one of these densely-connected layers) reduces this size now to the number of output classes, e.g. 2.

The densely-connected layer is followed by the fifteenth layer, which is a final activation layer 9. Here the activation function is specified as a “softmax” function, which again does not change the shape of the signal. This gives the final output of the network 1.

TABLE 1
Example description of the network architecture with example signal
sizes, the layers referring to the above description and FIG. 1.

| Layer | Name | Description | Input Size | Parameters | Output Size |
|---|---|---|---|---|---|
| 1 | 2D | Calculating a | (b, c, tp, 1) | Kernel size = (1, k) | (b, c, tp, f1) |
| | Convolution | convolution of the | | # Filters = f1 | |
| | | input with the kernel | | | |
| | | for f1 different filters | | | |
| 2 | Batch | Normalising all | (b, c, tp, f1) | | (b, c, tp, f1) |
| | Normalisation | intermediate results | | | |
| | | at this stage | | | |
| 3 | 2D | Calculating a | (b, c, tp, f1) | Kernel size = (c, 1) | (b, 1, tp, f1*d) |
| | Depthwise | convolution of the | | Depth Multiplier = d | |
| | Convolution | input with a kernel | | | |
| | | per filter separately | | | |
| 4 | Batch | See above | (b, 1, tp, f1*d) | | (b, 1, tp, f1*d) |
| | Normalisation | | | | |
| 5 | Activation | Applying an | (b, 1, tp, f1*d) | Activation | (b, 1, tp, f1*d) |
| | | activation function | | function = func | |
| | | to the signal | | | |
| 6 | Pooling | Calculating the | (b, 1, tp, f1*d) | Pool size = (1, 4) | (b, 1, tp/4, f1*d) |
| | | mean in an area of | | | |
| | | the “pool size”, and | | | |
| | | reducing the size of | | | |
| | | the signal | | | |
| 7 | Dropout | Randomly dropping | (b, 1, tp/4, f1*d) | Dropout rate = dr | (b, 1, tp/4, f1*d) |
| | | a percentage | | | |
| | | (dropout rate) of the | | | |
| | | signal (setting it to | | | |
| | | zero) | | | |
| 8 | 2D | Performed in two | (b, 1, tp/4, f1*d) | Kernel size = (1, 16) | (b, 1, tp/4, f2) |
| | Separable | steps, first as a two- | | Depth Multiplier = 1 | |
| | Convolution | dimensional | | # Filters = f2 | |
| | | depthwise | | | |
| | | convolution (see | | | |
| | | above) and then as | | | |
| | | a pointwise | | | |
| | | convolution | | | |
| | | between all filters | | | |
| | | and kernels | | | |
| 9 | Batch | See above | (b, 1, tp/4, f2) | | (b, 1, tp/4, f2) |
| | Normalisation | | | | |
| 10 | Activation | See above | (b, 1, tp/4, f2) | | (b, 1, tp/4, f2) |
| 11 | Pooling | See above | (b, 1, tp/4, f2) | Pool size = (1, 8) | (b, 1, tp/32, f2) |
| 12 | Dropout | See above | (b, 1, tp/32, f2) | Dropout rate = dr | (b, 1, tp/32, f2) |
| 13 | Flattening | Reducing the signal | (b, 1, tp/32, f2) | | (b, tp/32*f2) |
| | | to one dimension, | | | |
| | | by concatenation | | | |
| 14 | Dense | Multiplication of the | (b, tp/32*f2) | Output | (b, dout) |
| | | kernel with the input | | dimension = dout | |
| | | and summation | | | |
| | | over the second | | | |
| | | dimension | | | |
| 15 | Activation | See above | | Activation | (b, dout) |
| | | | | function = | |
| | | | | Softmax | |

TABLE 2
Network parameters and their example ranges, if applicable

| Parameter | | |
|---|---|---|
| Name | Description | Ranges |
| b | Batch Size; Number of | 64, but can also vary |
| | single-trials used to train the | |
| | network simultaneously | |
| c | Number of EEG electrodes | (5, 64) |
| | that were used to train the | |
| | network | |
| tp | Number of time points of the | 550, [−50 to 500 ms relative |
| | signal, = (length of recorded | to sound occurrence] |
| | single-trial) × (sample | |
| | frequency) | |
| f1 | Number of temporal filters | At least 16, no upper bound |
| k | Kernel Length of first | (8, 1024) |
| | convolutional layer; length of | |
| | the temporal filters | |
| d | Depth Multiplier, describing | At least 4, no upper bound |
| | how many filters per input | |
| | filter are used; Number of | |
| | spatial filters | |
| dr | Dropout rate, the percentage | (0.7, 0) |
| | of the signal that is randomly | |
| | dropped, i.e., set to zero | |
| f2 | The number of filters of the | At least 64, no upper bound |
| | pointwise convolution | |
| func | Type of activation function | ReLu or ELu |
| dout | Number of labels to predict | At least 2 (survivors/non- |
| | and therefore output | survivors), but can also be |
| | dimension of the network | up to 5 or even more, for |
| | | example corresponding to |
| | | the cerebral performance |
| | | category |
| Ir | Learning rate; specifying | (10*e−2, 10*e−7) |
| | how much the weights per | |
| | iteration of the training are | |
| | changed. | |

The training of the neural network 1 is done over multiple epochs. Per epoch the training data is run through the network once in a number of batches (of size b). The trainable weights of the network are updated per batch, based on the difference between the real labels and the predicted output of the network. The learning rate (Ir) sets how much these weights are updated at a time. The network 1 is not trained on all the available data, as some data is left on the side for later testing the network's performance on unseen data as an external validation. The data is totally split into three different sets: a training, validation and test set; where the first is used for training the network, the validation set is for evaluating independently the performance of the network during the training process and the test set is, as mentioned above used for evaluation of the process after the training. The validation set and the performance of the network on this set is used for the decision on stopping the training of the network, based on one or multiple metrics, such as for example the accuracy, binary cross entropy loss, among others.

The overall training of the network is done multiple times, by splitting the data into the different sets and then training and evaluating the network multiple times (e.g. 10), giving a number of networks (e.g. 10 networks with different parameter values but with the same network architecture). The selection of the final used network is again made based on the performance of the network on the validation set, based on one or multiple metrics, e.g., one of the above-mentioned metrics. The training, validation and test sets for example contain 60%, 20% and 20% of the patients, respectively.

The number of output classes (dout), for example 2 or 5, is selected depending on the required application. In the above example of predicting coma outcome, 2 output classes may correspond to survivors vs. non-survivors, while 5 output classes may correspond to the 5 Cerebral Performance Categories Scale, which evaluates long term neurological outcome.

The trained network is then used to create one final prediction (also referred to as a classification result) per patient, by combining multiple EEG single-trials sub-predictions of said patient, by for example averaging all of the network's predictions for these EEG single-trials. Thus, the network 1 issues one sub-prediction (also referred to as a sub-classification result) per patient per each EEG single-trial. For example, if the electrode set-up consists of 64 electrodes, the network would generate one sub-prediction across all 64 electrodes per sound. These sub-predictions are then combined to compute one prediction per patient, by calculating the mean over all sub-predictions, although other options are also possible, such as the median, or max/min prediction.

The number of EEG single-trials used for outcome prediction is typically 100 to 300, and should at least be 50 single-trials per patient. The types of sounds might be any or all of the four different sound types collected.

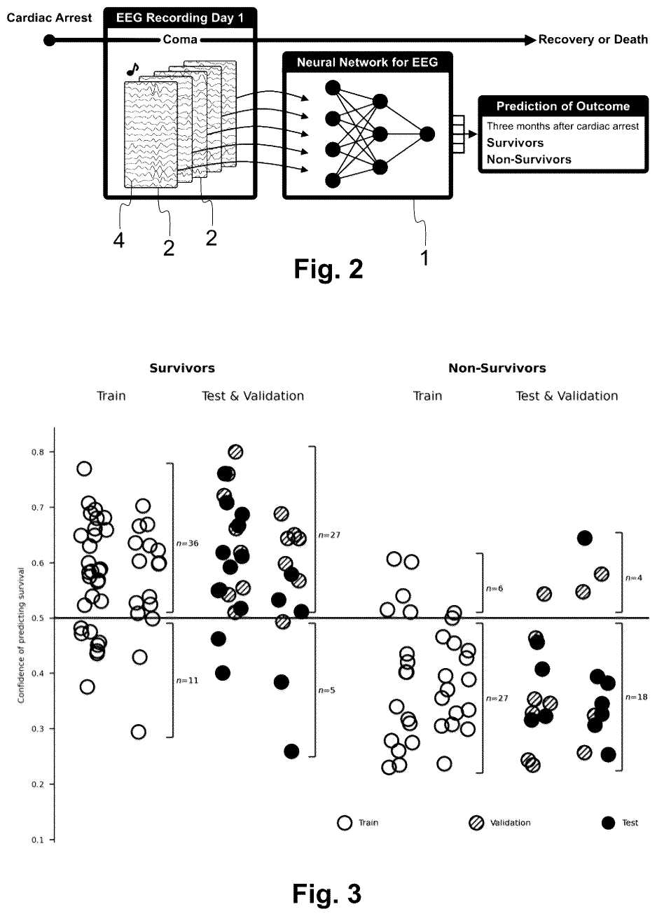

FIG. 2 schematically illustrates the above-described process of predicting outcome from coma with the use of the artificial neural network 1 and auditory stimulation. Auditory stimulation and EEG recordings are in this case performed on the first day of coma, shortly after a cardiac arrest. Single-trial EEG responses 2 (i.e., single-trials) are then given as input to the network 1, which classifies them as belonging to a survivor versus a non-survivor.

FIG. 3 shows some example results for predicting coma outcome based on the artificial neural network 1, in a group of 134 patients. Every circle corresponds to one patient. Data from patients of the training set (empty circles) were used to train the network. The prediction of outcome was performed on the test set of patients (full circles), whose data were never used to train or optimise the network. In FIG. 3, “Confidence of predicting survival” corresponds to the overall classification or prediction result per patient, and n denotes the number of patients by outcome and network's prediction. Predictive value for evaluating awakening from coma (among all patients predicted by the network as survivors, we quantify how many were actually survivors) was 0.92 in a test set of patients, and overall prediction of outcome (correctly predicted survivors and non-survivors) 0.83.

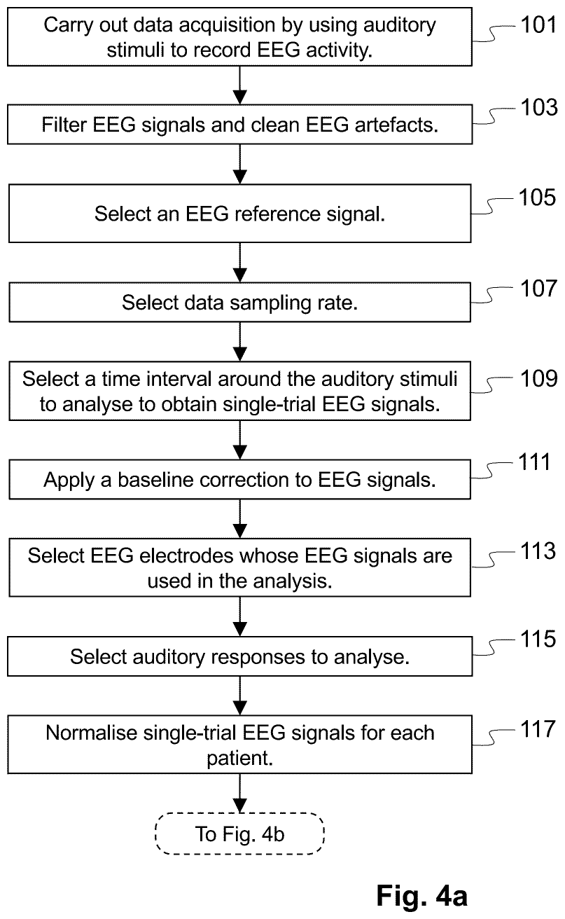

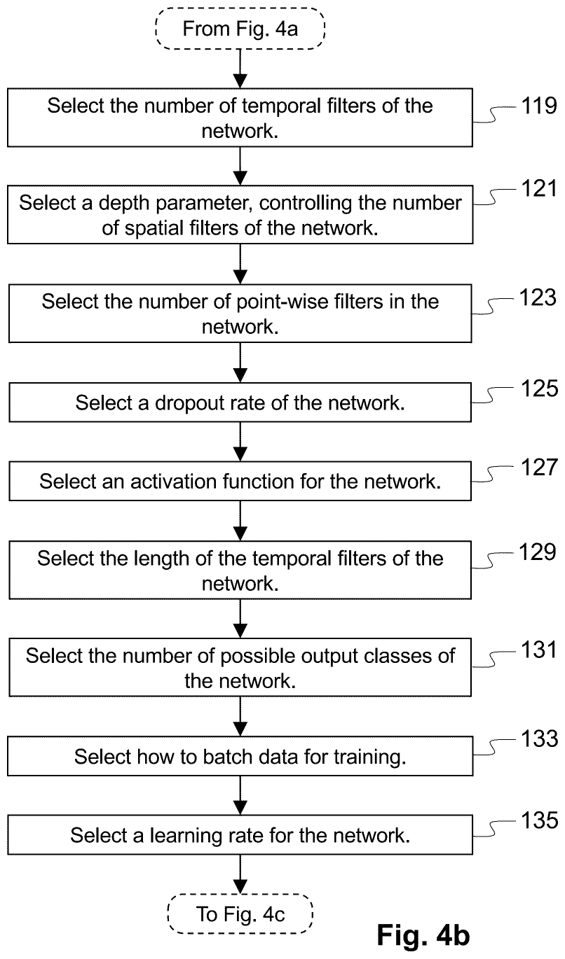

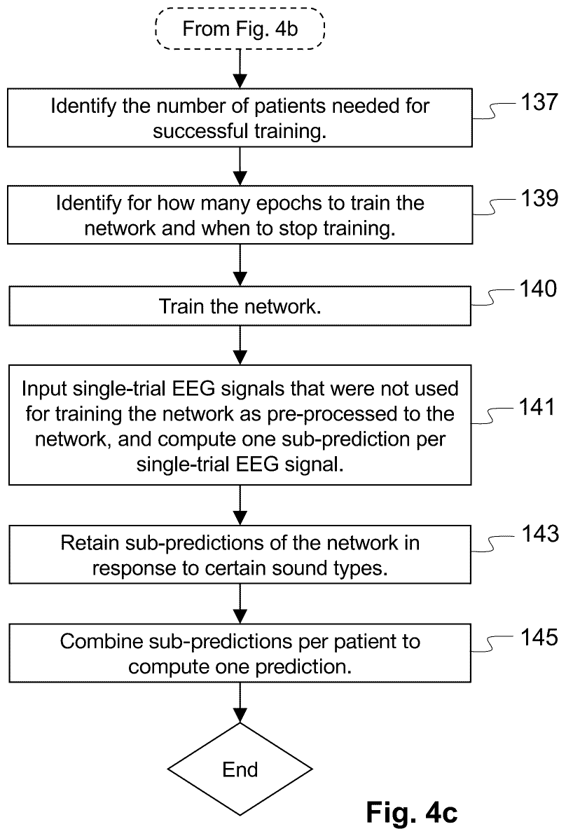

The flowchart of FIGS. 4a to 4c summarises the proposed data classification method according to one example when used to predict the survival of a post-anoxic comatose patient. It is to be noted that the steps of the method may be carried out in a different order than the one given in the flowchart. In step 101, the EEG signals are collected with scalp electrodes, during which patients are exposed to auditory stimulation. In other words, this step involves presenting post-anoxic coma patients with auditory stimuli at their bedside while recording their EEG activity. In step 103, the obtained EEG signals are filtered and cleaned from artefacts. In step 105, a reference signal is selected for the EEG signals to allow the EEG signals to be referenced to the reference signal. In step 107, a sampling rate is selected to sample the EEG signals with this sampling rate. In step 109, a time interval around the auditory stimuli is selected to obtain single-trial EEG signals, which are then used in the subsequent analysis. In step 111, a baseline correction is optionally applied to the EEG single-trials, where for every EEG single-trial the mean value of the interval before auditory stimulus presentation is calculated per electrode, and this value is then subtracted from every time point of the signal after the stimulus per electrode. In step 113, EEG electrodes are selected whose EEG signals are used in the subsequent analysis. In step 115, auditory responses to analyse are selected. In other words, some of the auditory responses may be discarded, for example if their quality is not sufficient. In step 117, the single-trial EEG signals for each patient are normalised, to a mean of zero and a standard deviation of one per single-trial. Steps 103 to 117 are part of a data preparation or pre-processing operation. These steps may be performed by a data processing apparatus automatically, or the user or operator may assist with at least some of the steps or independently perform some of the steps.

The architecture of the network is set next. In step 119, the number of temporal filters of the network is selected. In step 121, a depth parameter is selected, controlling the number of spatial filters of the network. In step 123, the number of pointwise filters in the network is selected. In step 125, a dropout rate of the network is selected. In step 127, an activation function for the network is selected. In step 129, the length of the temporal filters of the network is selected. The selections of steps 119 to 129 may be made by the operator, or the network may make the selections automatically based on predefined instructions.

The network is next set for the training. In step 131, the number of possible output classes of the network is selected. In step 133, batch data for training is selected. For example, the number of epochs per batch is selected. In step 135, a learning rate for the network is selected. In step 137, the number of patients needed for successful training is identified. In step 139, it is identified for how many epochs the network needs to be trained, and when to stop training. Steps 137 and 139 are carried out by inspecting the performance of the network during the training. In step 140, the network is then trained by using the parameter values or settings as selected in the previous steps. Steps 131 to 140 may be carried out or supervised by the operator. Alternatively, the network may make the selections automatically based on predefined instructions.

The outcome prediction by using the network is next carried out. In step 141, the network's predictions for each EEG single-trial are calculated, resulting in one sub-prediction per EEG single-trial. In step 143, the network's sub-predictions are retained over EEG single-trials in response to one or multiple sound types. In step 145, the sub-predictions are combined to compute one prediction about the patient's outcome. In this example the sub-predictions are combined over a plurality of sounds and optionally over a plurality of sound types. Thus, some of the deviant sound types (e.g., location and pitch) that were collected can be discarded. This may be useful in some applications, where it is not necessary to record all sound types, to decrease the time it takes to collect data. Furthermore, step 145 is in this example performed by a data processing unit other than the network 1, i.e., this step is performed outside the network architecture.

The teachings of the present invention may also be used to assess sleep-wake disorders. More specifically, the proposed analysis pipeline may in the future be used to differentiate patients with sleep-wake disorders. Similar to the description of the application to analyse comatose patients, in this case the sensors are electrodes of an electroencephalography (EEG) system, and the target group is formed of patients with a suspicion of a sleep-wake disorder. As part of the clinical routine, clinicians perform an overnight polysomnography recording, where among other signals patients' EEG is measured. Based on these measurements, we foresee that our developed method may be able to discriminate types of sleep-wake disorders, or sleep-wake disorders from healthy individuals. However, unlike the application on comatose patients this pipeline is not necessarily used within 24 hours and neither in the intensive care unit of hospitals.

The above-described method steps may be carried out by suitable circuits or circuitry. The terms “circuits” and “circuitry” refer to physical electronic components or modules (e.g. hardware), and any software and/or firmware (“code”) that may configure the hardware, be executed by the hardware, and or otherwise be associated with the hardware. The circuits may thus be operable to carry out or they comprise means for carrying out the required method steps as described above.

At least some of the method steps can be considered as computer-implemented steps. The invention also relates to a non-transitory computer program product comprising instructions for implementing the steps or at least some of the steps of the method when loaded and run on computing means of a computing device, such as the artificial neural network.

While the invention has been illustrated and described in detail in the drawings and foregoing description, such illustration and description are to be considered illustrative or exemplary and not restrictive, the invention being not limited to the disclosed embodiment. Other embodiments and variants are understood, and can be achieved by those skilled in the art when carrying out the claimed invention, based on a study of the drawings, the disclosure and the appended claims. New embodiments may be obtained by combining any of the teachings above.

In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A sensor system for analysing sensor signals obtained by a plurality of sensors connected to an object to be stimulated with a sequence of stimulation signals, the sensor system being configured to perform operations comprising:
acquire a set of sensor signals in response to stimulating a plurality of objects with a plurality of sequences of stimulation signals comprising a set of standard stimulation signals and a set of deviant stimulation signals;
pre-process the acquired set of sensor signals to obtain a set of pre-processed sensor signals, the pre-processing comprising at least the steps of cropping the sensor signals in the time domain timewise around the stimulation signals, filtering the sensor signals, and normalising the sensor signals;
prepare an artificial neural network for data analysis by using a first set of the pre-processed sensor signals, the preparation comprising the steps of training and validating the artificial neural network by using the first set of the pre-processed sensor signals, the artificial neural network comprising respective layers configured to perform temporal, spatial, pointwise, and depthwise convolutions, and the artificial neural network comprising adjustable parameters comprising at least learning rate, kernel lengths, filter lengths, dropout rate, and activation functions;
feed a second set of the pre-processed sensor signals to the artificial neural network as trained and validated; and
combine output signals of the artificial neural network as trained and validated in response to feeding the second set of the pre-processed sensor signals to the artificial neural network to obtain a single classification result for a respective object and for a respective sequence of stimulation signals.

2. The sensor system according to claim 1, wherein the second set of the pre-processed sensor signals consists of the sensor signals as pre-processed obtained from the plurality of sensors connected to the respective object for the respective sequence of stimulation signals.

3. The sensor system according to claim 1, wherein the stimulation signals are audio signals, and wherein the deviant stimulation signals are distinguished from the standard stimulation signals by their duration, pitch, and/or location, where location is in relation to the stimulated object.

4. The sensor system according to claim 1, wherein the sensors are electrodes of an electroencephalography system.

5. The sensor system according to claim 1, wherein the total number of layers in the artificial neural network is at least 13.

6. The sensor system according to claim 1, wherein the object is a patient, and wherein the classification results predict awakening of the patient from a coma.

7. The sensor system according to claim 6, wherein the classification result is the patient's coma outcome as survivor or non-survivor, or the patient's neurological long-term outcome, and.

8. The sensor system according to claim 1, wherein the layer configured to perform the temporal convolution comprises at least 16 filters, the layer configured to perform the spatial convolution has a depth multiplier of at least 4, and the layers configured to perform the pointwise and depthwise convolutions form a two-dimensional separable convolution having at least 64 filters.

9. The sensor system according to claim 1, wherein the artificial neural network as trained and validated is configured to generate one sub-classification result across the plurality of sensors per stimulation signal.

10. The sensor system according to claim 1, wherein the first set of the pre-processed sensor signals collectively form a training data set and a validation data set for the artificial neural network, while the second set of the pre-processed sensor signals forms a test data set for the artificial neural network.

11. An artificial neural network for analysing sensor signals obtained by a plurality of sensors connected to an object to be stimulated with a sequence of stimulation signals, the artificial neural network comprising at least 13 layers comprising:
a convolutional layer configured to convolve a respective sensor signal only along the temporal dimension, but not along the spatial dimension, the number of filters in the convolutional layer being equal to or greater than 16;
a two-dimensional depthwise convolutional layer characterised at least by a depth multiplier, and configured to reduce the size of a respective input signal of the two-dimensional depthwise convolutional layer, the depth multiplier having a value of at least 4;
a two-dimensional separable convolution layer having the number of filters equal to or greater than 64;
a batch normalisation layer, an activation layer, a pooling layer and/or dropout layer arranged between any of the convolutional layers;
a flattening layer configured to reduce a respective input signal of the flattening layer to one dimension;
at least one densely-connected layer having the number of output nodes equalling the number of possible output classes of the artificial neural network; and
an output activation layer forming an output layer of the artificial neural network, and configured to output a single classification result for a respective input signal of the artificial neural network.

12. The artificial neural network according to claim 11, wherein the convolutional layer has a first kernel of a length of at least 8.

13. The artificial neural network according to claim 11, wherein the two-dimensional depthwise convolution layer has a second kernel having a spatial dimension the same as the number of sensors connected to the object.

14. A method for analysing sensor signals obtained by a plurality of sensors, the method comprising:
acquiring a set of sensor signals in response to stimulating a plurality of objects with a plurality of sequences of stimulation signals comprising a set of standard stimulation signals and a set of deviant stimulation signals;
pre-processing the acquired set of sensor signals to obtain a set of pre-processed sensor signals, the pre-processing comprising at least the steps of cropping the sensor signals in the time domain timewise around the stimulation signals, filtering the sensor signals, and normalising the sensor signals;
preparing an artificial neural network for data analysis by using a first set of the pre-processed sensor signals, the preparation comprising the steps of training and validating the artificial neural network by using the first set of the pre-processed sensor signals, the artificial neural network comprising respective layers configured to perform temporal, spatial, pointwise, and depthwise convolutions, and the artificial neural network comprising adjustable parameters comprising at least learning rate, kernel lengths, filter lengths, dropout rate, and activation functions;
feeding a second set of the pre-processed sensor signals to the artificial neural network as trained and validated; and
combining output signals of the artificial neural network as trained and validated in response to feeding the second set of the pre-processed sensor signals to the artificial neural network to obtain a single classification result for a respective object and for a respective sequence of stimulation signals.

15. A computer program product comprising instructions for implementing the steps of the method of claim 14 when loaded and run on a computing apparatus.

16. The sensor system according to claim 1, wherein the pre-processing further comprises the step re-referencing the sensor signals to a reference signal.

17. The sensor system according to claim 4, wherein the number of electrodes per object is in the range of 5 to 64.

18. The sensor system according to claim 1, wherein the activation function for all the layers except the last one is either a rectified linear unit or exponential linear unit.

19. The sensor system according to claim 6, wherein the set of sensor signals are acquired within 24 hours from coma onset, following a cardiac arrest.

20. The sensor system according to claim 1, wherein the respective sequence of stimulation signals contains at least of 50 stimulation signals, and/or wherein the learning rate is at least 10*e-7, but not greater than 10*e-2.
